(19) United States
(12) Patent Application Publication
(10) Pub. No.: US 2010/0311207 A1
(43) Pub. Date: Dec. 9, 2010

(54) Compounds Having A Diphenyl Oxide Backbone and Maleimide Functional Group

(76) Inventor: Osama M. Musa, Hillsborough, NJ (US)

Correspondence Address: Henkel Corporation, 10 Finderne Avenue, Suite B, Bridgewater, NJ, 08807, US

(21) Appl. No.: 12/818,225

(22) Filed: Jun. 18, 2010

Related U.S. Application Data
(63) Continuation of application No. PCT/US2007/088130, filed on Dec. 19, 2007.

(51) Int. Cl. H01L 21/56; C07D 207/40; H01L 21/50

(52) U.S. Cl. 438/113; 548/547; 438/127; 257/E21.502; 257/E21.499

(57) ABSTRACT

A compound having a diphenyl oxide backbone, and pendant from the backbone at least one hydrocarbon chain, the hydrocarbon chain containing an ester functionality and being terminated with a maleimide functional group is prepared from the reaction of diphenyl oxide, formaldehyde or paraformaldehyde, and a compound containing both carboxylic acid and maleimide functionality. Exemplary compounds include:

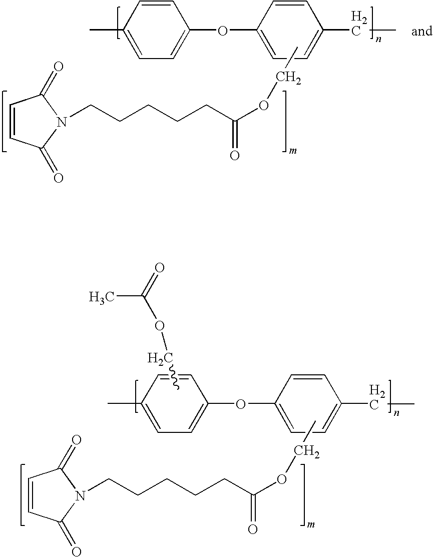

in which m and n are independently an integer from 1 to 100, provided that n is greater than m.

### CROSS-REFERENCE TO RELATED APPLICATION

This application is a continuation of International Application No. PCT/US2007/088130 filed Dec. 19, 2007.

### FIELD OF THE INVENTION

This invention relates to compounds having a diphenyl oxide backbone, which compounds are useful as adhesives, coatings, and encapsulants. These compounds are particularly useful for various fabrication steps in semiconductor packaging.

### BACKGROUND OF THE INVENTION

Adhesives for use on metal, glass, and plastic surfaces have many applications within various industries. Adhesion to these surfaces in general is difficult and new compounds or formulations are sought for both quick and strong adherence. Such compounds would be particularly useful within the semiconductor packaging industry. Common steps in the fabrication of semiconductor packages involve affixing semiconductor devices onto substrates or encapsulating or coating parts, or all, of the device. The more prominent steps that use adhesives, coatings or encapsulants are the bonding of integrated circuit chips to lead frames or other substrates, the bonding of circuit packages or assemblies to printed wire boards, the encapsulation of solder balls used as electrical connections, and the coating of via holes. In these applications, the components of the package are prepared from different materials, such as metal, glass, silicon, and plastic, and the adhesive or encapsulant must bond to the surface of each. Moreover, the adhesive or encapsulant must maintain its bond to both materials through temperature and humidity cycles. Thus, there is always a need for new compounds and formulations to provide good adhesion to a variety of surfaces, within the semiconductor packaging industry and within other industries using components that must adhere to a surface.

### SUMMARY OF THE INVENTION

This invention is a compound having a diphenyl oxide (DPO) backbone, and pendant from the backbone at least one hydrocarbon chain, the hydrocarbon chain containing an ester functionality and being terminated with a maleimide functional group. The compound may further have pendant from the backbone at least one additional hydrocarbon chain, the additional hydrocarbon chain containing an ester functionality and being terminated with a group other than a maleimide. In other embodiments, this invention is a curable composition containing the inventive compound; an article having deposited thereon the inventive compound or curable composition; and a process for making a semiconductor device using the inventive compound or curable composition.

### DETAILED DESCRIPTION OF THE INVENTION

In one embodiment, this invention is a compound having a diphenyl oxide (DPO) backbone, and pendant from the backbone at least one hydrocarbon chain, the hydrocarbon chain containing an ester functionality and being terminated with a maleimide functional group. The compound may further have pendant from the backbone at least one additional hydrocarbon chain, the additional hydrocarbon chain containing an ester functionality and being terminated with a group other than a maleimide. The group other than the maleimide may be either reactive or non-reactive. By reactive is meant that the group is capable of reacting with another organic compound to form a new covalent bond, for example, such as can be formed by the reaction of two carbon to carbon double bonds under free radical initiation, or by a ring opening mechanism of an epoxide, oxetane, triazole, cyanurate, or oxazoline. If more than one additional group, other than the maleimide, is present, those groups may be all reactive, all non-reactive, or a combination of reactive and non-reactive. In one embodiment the additional functional group is selected from the group consisting of acrylate, methacrylate, maleate, fumarate, styrenic, cinnamyl, or any combination of these.

In another embodiment, this invention is a curable composition comprising one or more other curable resins in addition to the compound having the diphenyl oxide backbone and maleimide functionality, with or without a group other than the maleimide functionality. The additional curable resin may be the major component or just a minor component of the curable composition. The curable composition may also comprise curing agents, adhesion promoters, fillers, wetting agents, fluxing agents, and other such components commonly used in curable compositions. Curable compositions are used, for example, in adhesive, coating, and encapsulation formulations.

In a further embodiment, this invention is an article, for example a semiconductor device, on which has been deposited the DPO compound or curable composition containing the DPO compound.

In another embodiment this invention is a process for making a semiconductor device comprising the steps of (i) providing a semiconductor wafer having a front side that is active and opposed to the front side a back side that is inactive, (ii) providing a curable composition comprising a compound having a diphenyl oxide backbone and pendant from the backbone at least one hydrocarbon chain, the hydrocarbon chain containing an ester functionality and being terminated with a maleimide functional group, (iii) applying the curable composition to the back side of the semiconductor wafer to form an adhesive layer having an exposed side opposite the front side of the semiconductor wafer, (iv) B-staging the adhesive layer to form a B-staged wafer, (v) dicing the B-staged wafer into at least one semiconductor die having an adhesive layer on the back side of the semiconductor die, (vi) contacting the semiconductor die to a substrate such that the adhesive layer is disposed between the semiconductor die and the substrate, and (vii) curing the adhesive for a time and temperature to adhere the semiconductor die to the substrate to form a semiconductor device. The DPO compound may contain groups other than the maleimide as described above, and the curable composition may contain other resins.

The term “B-staging” (and its variants) is used to refer to the processing of a material by heat or irradiation so that if the material is dissolved or dispersed in a solvent, the solvent is evaporated off with or without partial curing of the material, or if the material is neat with no solvent, the material is partially cured to a tacky or more hardened state. If the material is a flow-able adhesive, B-staging will provide extremely low flow without fully curing, such that additional curing may be performed after the adhesive is used to join one article to another. The reduction in flow may be accomplished by evaporation of a solvent, partial advancement or curing of a resin or polymer, or both.

In a further embodiment, this invention is a process for underfilling a flip chip semiconductor device comprising the steps of (i) providing a flip chip die that has been attached to a substrate such that there is a gap between the flip chip die and the substrate, (ii) dispensing a curable composition comprising a compound having a diphenyl oxide backbone and pendant from the backbone at least one hydrocarbon chain, the hydrocarbon chain containing an ester functionality and being terminated with a maleimide functional group, onto the substrate along at least one side of the flip chip die, (iii) allowing the curable composition to flow into the gap between the flip chip die and the substrate, and (iv) curing the curable composition. The DPO compound may contain groups other than the maleimide as described above, and the curable composition may contain other resins.

In an additional embodiment, this invention is a process for attaching a semiconductor die to a substrate comprising the steps of (i) providing a semiconductor die, a substrate, and a curable composition comprising a compound having a diphenyl oxide backbone and pendant from the backbone at least one hydrocarbon chain, the hydrocarbon chain containing an ester functionality and being terminated with a maleimide functional group, (ii) applying the curable composition to the substrate, the die, or both, (iii) joining the semiconductor die to the substrate with the curable composition disposed between them, and (iv) curing the curable composition. The DPO compound may contain groups other than the maleimide as described above, and the curable composition may contain other resins.

The compound having a diphenyl oxide backbone and pendant from the backbone at least one hydrocarbon chain, the hydrocarbon chain containing an ester functionality and being terminated with a maleimide functional group, is prepared from the reaction of diphenyl oxide, formaldehyde or paraformaldehyde, and a compound containing both carboxylic acid and maleimide functionality. If groups other than the maleimide are desired, the reaction mix will further contain a compound containing carboxylic acid and the group other than the maleimide.

The compound containing both carboxylic acid and maleimide functionality will have a hydrocarbon chain linking the acid and maleimide functionality, and the compound containing both carboxylic acid and a group other than the maleimide, will have a hydrocarbon chain linking the acid and group other than the maleimide functionality. The hydrocarbon chain in either case will typically contain linear methylene groups forming the chain, although the chain may have cyclic aliphatic groups, aromatic groups, or heteroatoms incorporated into the chain. The length of the chain can be varied to design specific molecular weights and performance properties and is limited only by the availability or synthetic feasibility of the starting carboxylic acid containing the maleimide functionality or group other than maleimide. A higher molecular weight hydrocarbon chain pendant from the backbone will give higher modulus at elevated temperatures. The groups other than the maleimide, also pendant from the DPO backbone, will give specific properties depending on the choice of the practitioner as one skilled in the art would know how to choose.

The DPO backbone may also be tailored to suit the needs of the practitioner. For instance, a longer DPO backbone will provide enhanced toughening properties.

The preparation of the compound can be carried out using well-known free radical polymerization procedures, using solution, emulsion, or bulk polymerization techniques. The compound is formed by removal of the solvent, coagulation of the latex, or melt-processing of the neat polymer.

The compound of this invention may form a curable composition either by itself or combined with other components. The curable composition that comprises only the compound of this invention cures upon the application of heat, which causes initiation of free-radical curing. Alternatively, the curable composition may be formulated to include, in addition to the inventive compound, other resins or polymers, fillers, and additives including curing agents, adhesion promoters, fluxing agents, anti-foaming agents, solvents, and the like. Resins and polymers used in the formulation, in addition to the inventive compound, may be solid, liquid, or a combination of the two. Suitable additional resins and polymers include but are not limited to epoxies, acrylates and methacrylates, maleimides, bismaleimides, vinyl ethers, polyesters, poly(butadienes), siliconized olefins, silicone resins, siloxanes, styrene resins and cyanate ester resins.

Exemplary solid aromatic bismaleimide (BMI) resin powders for use in formulations with the inventive compounds are those having the structure

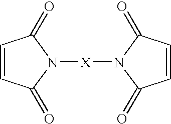

in which X is an aromatic group. Bismaleimide resins having these X bridging groups are commercially available, and can be obtained, for example, from Sartomer (USA) or HOS-Technic GmbH (Austria).

Additional exemplary maleimide resins for use in formulations with the inventive compounds include those having the generic structure

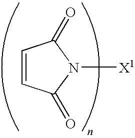

in which n is 1 to 3 and X¹ is an aliphatic or aromatic group. Exemplary X¹ entities include, poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. These types of resins are commercially available and can be obtained, for example, from National Starch and Chemical Company and Dainippon Ink and Chemical, Inc.

Specific preferred maleimide resins include

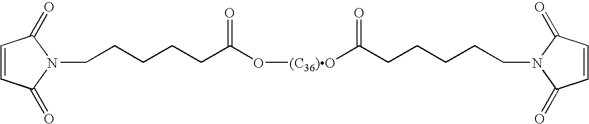

in which C₃₆ represents a linear or branched chain (with or without cyclic moieties) of 36 carbon atoms;

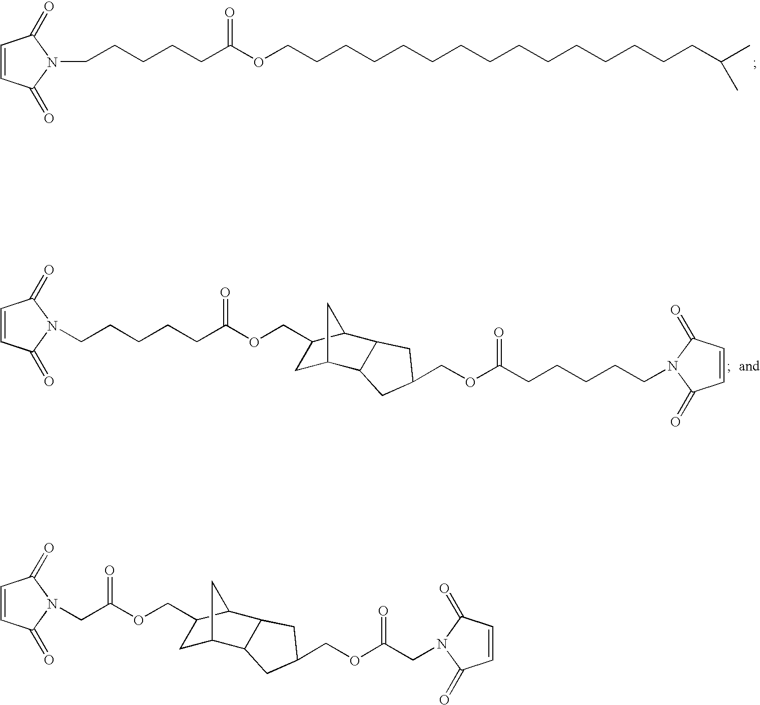

Suitable acrylate resins for use in formulation with the inventive compounds include those having the generic structure

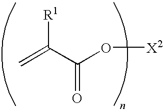

in which n is 1 to 6, R¹ is —H or —CH₃. and X² is an aromatic or aliphatic group. Exemplary X² entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. Commercially available materials include butyl (meth)acrylate, isobutyl(meth)acrylate, 2-ethyl hexyl(meth)acrylate, isodecyl (meth)acrylate, n-lauryl(meth)acrylate, alkyl(meth)acrylate, tridecyl(meth)acrylate, n-stearyl (meth)acrylate, cyclohexyl(meth)acrylate, tetrahydrofurfuryl(meth)acrylate, 2-phenoxy ethyl(meth)acrylate, isobornyl(meth)acrylate, 1,4-butanediol di(meth)acrylate, 1.6 hexanediol di(meth)acrylate, 1,9-nonandiol di(meth)acrylate, perfluorooctylethyl (meth)acrylate, 1,10 decandiol di(meth)acrylate, nonylphenol polypropoxylate (meth)acrylate, and polypentoxylate tetrahydrofurfuryl acrylate, available from Kyoeisha Chemical Co., LTD; polybutadiene urethane dimethacrylate (CN302, NTX6513) and polybutadiene dimethacrylate (CN301, NTX6039, PRO6270) available from Sartomer Company, Inc; polycarbonate urethane diacrylate (ArtResin UN9200A) available from Negami Chemical Industries Co., LTD; acrylated aliphatic urethane oligomers (Ebecryl 230, 264, 265, 270, 284, 4830, 4833, 4834, 4835, 4866, 4881, 4883, 8402, 8800-20R, 8803, 8804) available from Radcure Specialities, Inc; polyester acrylate oligomers (Ebecryl 657, 770, 810, 830, 1657, 1810, 1830) available from Radcure Specialities, Inc.; and epoxy acrylate resins (CN104, 111, 112, 115, 116, 117, 118, 119, 120, 124, 136) available from Sartomer Company, Inc. In one embodiment the acrylate resins are selected from the group consisting of isobornyl acrylate, isobornyl methacrylate, lauryl acrylate, lauryl methacrylate, poly(butadiene) with acrylate functionality and poly(butadiene) with methacrylate functionality.

Suitable vinyl ether resins for use in formulations with the inventive compounds include those having the generic structure

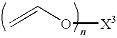

in which n is 1 to 6 and X³ is an aromatic or aliphatic group. Exemplary X³ entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. Commercially available resins include cyclohenanedimethanol divinylether, dodecylvinylether, cyclohexyl vinylether, 2-ethylhexyl vinylether, dipropyleneglycol divinylether, hexanediol divinylether, octadecylvinylether, and butandiol divinylether available from International Speciality Products (ISP); Vectomer 4010, 4020, 4030, 4040, 4051, 4210, 4220, 4230, 4060, 5015 available from Sigma-Aldrich, Inc.

Suitable poly(butadiene) resins for use in formulations with the inventive compounds include poly(butadienes), epoxidized poly(butadienes), maleic poly(butadienes), acrylated poly(butadienes), butadiene-styrene copolymers, and butadiene-acrylonitrile copolymers. Commercially available materials include homopolymer butadiene (Ricon130, 131, 134, 142, 150, 152, 153, 154, 156, 157, P30D) available from Sartomer Company, Inc; random copolymer of butadiene and styrene (Ricon 100, 181, 184) available from Sartomer Company Inc.; maleinized poly(butadiene) (Ricon 130MA8, 130MA13, 130MA20, 131MA5, 131MA10, 131MA17, 131MA20, 156MA17) available from Sartomer Company, Inc.; acrylated poly(butadienes) (CN302, NTX6513, CN301, NTX6039, PRO6270, Ricacryl 3100, Ricacryl 3500) available from Sartomer Inc.; epoxydized poly(butadienes) (Polybd 600, 605) available from Sartomer Company. Inc. and Epolead PB3600 available from Daicel Chemical Industries, Ltd; and acrylonitrile and butadiene copolymers (Hycar CTBN series, ATBN series, VTBN series and ETBN series) available from Hanse Chemical.

Suitable epoxy resins for use in formulations containing the inventive compounds include bisphenol, naphthalene, and aliphatic type epoxies. Commercially available materials include bisphenol type epoxy resins (Epiclon 830LVP, 830CRP, 835LV, 850CRP) available from Dainippon Ink & Chemicals, Inc.; naphthalene type epoxy (Epiclon HP4032) available from Dainippon Ink & Chemicals, Inc.; aliphatic epoxy resins (Araldite CY179, 184, 192, 175, 179) available from Ciba Specialty Chemicals, (Epoxy 1234, 249, 206) available from Union Carbide Corporation, and (EHPE-3150) available from Daicel Chemical Industries, Ltd. Other suitable epoxy resins include cycloaliphatic epoxy resins, bisphenol-A type epoxy resins, bisphenol-F type epoxy resins, epoxy novolac resins, biphenyl type epoxy resins, naphthalene type epoxy resins, dicyclopentadiene-phenol type epoxy resins, reactive epoxy diluents, and mixtures thereof.

Suitable siliconized olefin resins for use in the formulations containing the inventive compounds are obtained by the selective hydrosilation reaction of silicone and divinyl materials, having the generic structure,

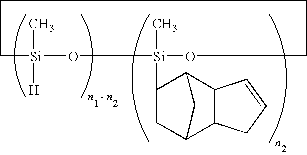

in which n₁ is 2 or more, n₂ is 1 or more and n₁>n₂. These materials are commercially available and can be obtained, for example, from National Starch and Chemical Company.

Suitable silicone resins for use in formulations with the inventive compounds include reactive silicone resins having the generic structure

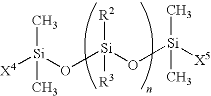

in which n is 0 or any integer, X⁴ and X⁵ are hydrogen, methyl, amine, epoxy, carboxyl, hydroxy, acrylate, methacrylate, mercapto, phenol, or vinyl functional groups, R² and R³ can be —H, —CH₃, vinyl, phenyl, or any hydrocarbon structure with more than two carbons. Commercially available materials include KF8012, KF8002, KF8003, KF-1001, X-22-3710, KF6001, X-22-164C, KF2001, X-22-170DX, X-22-173DX, X-22-174DX X-22-176DX, KF-857, KF862, KF8001, X-22-3367, and X-22-3939A available from Shin-Etsu Silicone International Trading (Shanghai) Co., Ltd.

Suitable styrene resins for use in formulations with the inventive compounds include those resins having the generic structure

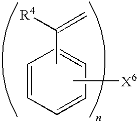

in which n is 1 or greater, R⁴ is —H or —CH₃, and X⁶ is an aliphatic group. Exemplary X³ entities include poly(butadienes), poly(carbonates), poly(urethanes), poly(ethers), poly(esters), simple hydrocarbons, and simple hydrocarbons containing functionalities such as carbonyl, carboxyl, amide, carbamate, urea, or ether. These resins are commercially available and can be obtained, for example, from National Starch and Chemical Company or Sigma-Aldrich Co.

Suitable cyanate ester resins for use in formulations with the inventive compounds include those having the generic structure

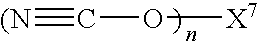

in which n is 1 or larger, and X⁷ is a hydrocarbon group. Exemplary X⁷ entities include bisphenol, phenol or cresol novolac, dicyclopentadiene, polybutadiene, polycarbonate, polyurethane, polyether, or polyester. Commercially available materials include; AroCy L-10, AroCy XU366, AroCy XU371, AroCy XU378, XU71787.02L, and XU 71787.07L, available from Huntsman LLC; Primaset PT30, Primaset PT30 S75, Primaset PT60, Primaset PT60S, Primaset BADCY, Primaset DA230S, Primaset MethylCy, and Primaset LECY, available from Lonza Group Limited; 2-allyphenol cyanate ester, 4-methoxyphenol cyanate ester, 2,2-bis(4-cyanatophenol)-1,1,1,3,3,3-hexafluoropropane, bisphenol A cyanate ester, diallylbisphenol A cyanate ester, 4-phenylphenol cyanate ester, 1,1,1-tris(4-cyanatophenyl)ethane, 4-cumylphenol cyanate ester, 1,1-bis(4-cyanateophenyl)ethane, 2,2,3,4,4,5,5,6,6,7,7-dodecafluorooctanediol dicyanate ester, and 4,4′-bisphenol cyanate ester, available from Oakwood Products, Inc.

Additional polymers suitable for use in formulations with the inventive compounds include polyamide, phenoxy, polybenzoxazine, polyether sulfone, polyimide, benzoxazine, vinyl ether, polyolefin, polybenzoxyzole, polyester, polystyrene, polycarbonate, polypropylene, poly(vinyl chloride), polyisobutylene, polyacrylonitrile, poly(methyl methacrylate), poly(vinyl acetate), poly(2-vinylpridine), cis-1,4-polyisoprene, 3,4-polychloroprene, vinyl copolymer, poly(ethylene oxide), poly(ethylene glycol), polyformaldehyde, polyacetaldehyde, poly(b-propiolacetone), poly(10-decanoate), poly(ethylene terephthalate), polycaprolactam, poly(11-undecanoamide), poly(m-phenylene-terephthalamide), poly(tetramethlyene-m-benzenesulfonamide), polyester polyarylate, poly(phenylene oxide), poly(phenylene sulfide), polysulfone, polyimide, polyetheretherketone, polyetherimide, fluorinated polyimide, polyimide siloxane, poly-iosindolo-quinazolinedione, polythioetherimide poly-phenyl-quinoxaline, polyquuinixalone, imide-aryl ether phenylquinoxaline copolymer, polyquinoxaline, polybenzimidazole, polybenzoxazole, polynorbornene, poly(arylene ethers), polysilane, parylene, benzocyclobutenes, hydroxy(benzoxazole) copolymer, poly(silarylene siloxanes), and polybenzimidazole.

Other suitable materials for inclusion in adhesive, coating, and encapsulant compositions containing the inventive compounds include rubber polymers such as block copolymers of monovinyl aromatic hydrocarbons and conjugated diene, e.g., styrene-butadiene, styrene-butadiene-styrene (SBS), styrene-isoprene-styrene (SIS), styrene-ethylene-butylene-styrene (SEBS), and styrene-ethylene-propylene-styrene (SEPS).

Other suitable materials for inclusion in compositions containing the inventive compounds include ethylene-vinyl acetate polymers, other ethylene esters and copolymers, e.g., ethylene methacrylate, ethylene n-butyl acrylate and ethylene acrylic acid; polyolefins such as polyethylene and polypropylene; polyvinyl acetate and random copolymers thereof; polyacrylates; polyamides; polyesters; and polyvinyl alcohols and copolymers thereof.

Suitable thermoplastic rubbers for use in formulations containing the inventive compounds include carboxy terminated butadiene-nitrile (CTBN)/epoxy adduct, acrylate rubber, vinyl-terminated butadiene rubber, and nitrile butadiene rubber (NBR). In one embodiment the CTBN epoxy adduct consists of about 20-80 wt % CTBN and about 20-80 wt % diglycidyl ether bisphenol A: bisphenol A epoxy (DGEBA). A variety of CTBN materials are available from Noveon Inc., and a variety of bisphenol A epoxy materials are available from Dainippon Ink and Chemicals, Inc., and Shell Chemicals. NBR rubbers are commercially available from Zeon Corporation.

Suitable siloxanes for use in formulations containing the inventive compounds include elastomeric polymers comprising a backbone and pendant from the backbone at least one siloxane moiety that imparts permeability, and at least one reactive moiety capable of reacting to form a new covalent bond. Examples of suitable siloxanes include elastomeric polymers prepared from: 3-(tris(trimethyl-silyloxy)silyl)-propyl methacrylate, n-butyl acrylate, glycidyl methacrylate, acrylonitrile, and cyanoethyl acrylate; 3-(tris(trimethylsilyloxy)silyl)-propyl methacrylate, n-butyl acrylate, glycidyl methacrylate, and acrylonitrile; and 3-(tris(trimethylsilyloxy)silyl)-propyl methacrylate, n-butyl acrylate, glycidyl methacrylate, and cyanoethyl acrylate.

A curing agent may be used to initiate, propagate, catalyze, accelerate, or otherwise facilitate the curing of the inventive compound and/or of any additional resins or polymers included in the formulation. Selection of a curing agent is dependent on the inventive compound, resins, and polymers used and the processing conditions employed. As curing agents, the compositions may use aromatic amines, alycyclic amines, aliphatic amines, tertiary phosphines, triazines, metal salts, aromatic hydroxyl compounds, or a combination of these. Appropriateness of the type and amount of curing agents used for specific compositions is disclosed in the open literature and is within the expertise of one skilled in the art.

Examples of such curing agents include imidazoles, such as 2-methylimidazole, 2-undecylimidazole, 2-heptadecyl imidazole, 2-phenylimidazole, 2-ethyl 4-methylimidazole, 1-benzyl-2-methylimidazole, 1-propyl-2-methylimidazole, 1-cyanoethyl-2-methylimidazole, 1-cyanoethyl-2-ethyl-4-methylimidazole, 1-cyanoethyl-2-undecylimidazole, 1-cyanoethyl-2-phenylimidazole, 1-guanaminoethyl-2-methylimidazole and addition product of an imidazole and trimellitic acid; tertiary amines, such as N,N-dimethyl benzylamine, N,N-dimethylaniline, N,N-dimethyltoluidine, N,N-dimethyl-p-anisidine, p-halogeno-N,N-dimethylaniline, 2-N-ethylanilino ethanol, tri-n-butylamine, pyridine, quinoline, N-methylmorpholine, triethanolamine, triethylenediamine, N,N,N′,N′-tetramethylbutanediamine, N-methylpiperidine; phenols, such as phenol, cresol, xylenol, resorcine, and phloroglucin; organic metal salts, such as lead naphthenate, lead stearate, zinc naphthenate, zinc octolate, tin oleate, dibutyl tin maleate, manganese naphthenate, cobalt naphthenate, and acetyl aceton iron; and inorganic metal salts, such as stannic chloride, zinc chloride and aluminum chloride; peroxides, such as benzoyl peroxide, lauroyl peroxide, octanoyl peroxide, acetyl peroxide, para-chlorobenzoyl peroxide and di-t-butyl diperphthalate; acid anhydrides, such as carboxylic acid anhydride, maleic anhydride, phthalic anhydride, lauric anhydride, pyromellitic anhydride, trimellitic anhydride, hexahydrophthalic anhydride; hexahydropyromellitic anhydride and hexahydrotrimellitic anhydride, azo compounds, such as azoisobutylonitrile, 2,2′-azobispropane, m,m′-azoxystyrene, hydrozones, and mixtures thereof.

Suitable curing accelerators may be selected from the group consisting of triphenylphosphine, alkyl-substituted imidazoles, imidazolium salts, onium salts, quartenary phosphonium compounds, onium borates, metal chelates, 1,8-diazacyclo[5.4.0]undex-7-ene or a mixture thereof.

Curing agents for the inventive compound must be a free radical initiator. Curing agents for additional resins or polymers in the composition can be either a free radical initiator or cationic initiator, depending on whether a radical or ionic curing resins are chosen. If a free radical initiator is used, it will be present in an effective amount. An effective amount typically is 0.1 to 10 percent by weight of the organic compounds (excluding any filler). Appropriate free-radical initiators include peroxides, such as butyl peroctoates and dicumyl peroxide, and azo compounds, such as 2,2′-azobis(2-methyl-propanenitrile) and 2,2′-azobis(2-methyl-butanenitrile). Preferred cationic curing agents include dicyandiamide, phenol novolak, adipic dihydrazide, diallyl melamine, diamino malconitrile, BF3-amine complexes, amine salts and modified imidazole compounds.

Metal compounds also can be employed as cure accelerators for cyanate ester systems and include, but are not limited to, metal napthenates, metal acetylacetonates (chelates), metal octoates, metal acetates, metal halides, metal imidazole complexes, and metal amine complexes. Other cure accelerators that may be included in the coating formulation include triphenylphosphine, alkyl-substituted imidazoles, imidazolium salts, and onium borates

In some cases, it may be desirable to use more than one type of cure. For example, both cationic and free radical initiation may be desirable, in which case both free radical cure and ionic cure resins can be used in the composition. These compositions would contain effective amounts of initiators for each type of resin. Such a composition would permit, for example, the curing process to be started by cationic initiation using UV irradiation, and in a later processing step, to be completed by free radical initiation upon the application of heat.

If the curable composition contains solvent it will typically require a drying and/or B-staging step. As used herein, “B-staging” (and its variants) is used to refer to the processing of a material by heat or irradiation so that if the material is solubilized or dispersed in a solvent, the solvent is evaporated off with or without partial curing of the material, or if the material is neat with no solvent, the material is partially cured to a tacky or more hardened state. For example, if the material is a flow-able adhesive, B-staging will provide extremely low flow without fully curing, such that additional curing may be performed after the adhesive is used to join one article to another. The reduction in flow may be accomplished by evaporation of a solvent, partial advancement or curing of a resin or polymer, or both. The time and temperature required to achieve this will vary according to the solvent and composition used and can be determined by the practitioner without undue experimentation. The drying and/or B-staging may be done as a step separate from the curing of the end use composition, or it may be done as a separate process step.

If the composition does not contain solvent it may still be desirable to B-stage, or partially advance, the material. This may be done prior to cure to effect hardening of the curable composition to a non-tacky state so that additional processing may be done before the curable composition is fully cured.

The curable composition may be cured either in an individual process step or in conjunction with another processing operation such as wirebonding or solder reflow. When the curable composition is used on semiconductor die, the cure may be done at the wafer level or at the die level, depending on the purpose of the composition, the makeup of the composition, and the manufacturing process employed.

Heat curing of the curable composition will generally take place within a range of 80°-250° C., and curing will be effected within a time period ranging from few seconds up to 120 minutes, depending on the particular compound and curing agents chosen. The time and temperature curing profile for each composition will vary, and different compositions can be designed to provide the curing profile that will be suited to the particular industrial manufacturing process.

Depending on the end application, one or more fillers or spacers, or both, may be included in the curable composition and usually are added for improved rheological properties, stress reduction, and bondline control. Examples of suitable nonconductive fillers include alumina, aluminum hydroxide, silica, vermiculite, mica, wollastonite, calcium carbonate, titania, sand, glass, barium sulfate, zirconium, carbon black, organic fillers, and halogenated ethylene polymers, such as, tetrafluoroethylene, trifluoroethylene, vinylidene fluoride, vinyl fluoride, vinylidene chloride, and vinyl chloride. Exemplary electrically or thermally conductive fillers include carbon black, graphite, gold, silver, copper, platinum, palladium, nickel, aluminum, silicon carbide, boron nitride, diamond, and alumina. The particles may be of any appropriate size ranging from nano size to several mm, depending on whether they are used as fillers or spcers (spacers typically being the larger size particles with more uniformity of size). The choice of such size for any particular end use is within the expertise of one skilled in the art. Filler may be present in any amount determined by the practitioner to be suitable for the chosen end use.

It is desirable for some curable compositions to add a fluxing agent to remove metal oxides and prevent re-oxidation of electrical solder joints or of metallic substrates. Fluxing agent selection will depend on the resin chemistry and metallurgy presented. Some of the key requirements of the fluxing agent are that it, and fluxing residues generated by the fluxing process, should not affect the curing of the inventive compounds, polymers, or resins present in the composition, should not be too corrosive, and should not out-gas to a detrimental level during heating cycles.

Examples of suitable fluxing agents include compounds that contain one or more hydroxyl groups (—OH), or carboxylic (—COOH) groups or both, such as are present in organic carboxylic acids, anhydrides, and alcohols. Exemplary fluxing agents are, for example, rosin gum, dodecanedioic acid (commercially available as Corfree M2 from Aldrich), sebacic acid, polysebasic polyanhydride, maleic acid, hexahydrophthalic anhydride, methyl hexahydrophthalic anhydride, ethylene glycol, glycerin, tartaric acid, adipic acid, citric acid, malic acid, glutaric acid, glycerol, 3-[bis(glycidyl oxy methyl)methoxy]-1,2-propane diol, D-ribose, D-cellobiose, cellulose, 3-cyclo-hexene-I,1-dimethanol; amine fluxing agents, such as, aliphatic amines having 1 to 10 carbon atoms, e.g., trimethylamine, triethylamine, n-propylamine, n-butylamine, isobutylamine, sec-butylamine, t-butylamine, n-amylamine, sec-amylamine, 2-ethylbutylamine, n-heptylamine, 2-ethylhexylamine, n-octylamine, and t-octylamine; and epoxy resins employing a cross-linking agent with fluxing properties. Fluxing agents may also be compounds that chelate with a metal substrate. Fluxing agents will be present in an effective amount, and typically an effective amount ranges from 1 to 30% by weight (excluding any filler content).

In some curable compositions it may be desirable to include a coupling agent. Suitable coupling agents are epoxy silanes, amine silanes agent, or mercapto silanes. Coupling agents, if used, will be used in an effective amount, and a typical effective amount is an amount up to 5% by weight (excluding any filler content).

For some applications, the curable composition may also contain a surfactant. Suitable surfactants include organic acrylic polymers, silicones, polyethylene glycol, polyoxyethylene/polyoxypropylene block copolymers, ethylene diamine based polyoxyethylene/polyoxypropylene block copolymers, polyol-based polyoxyalkylenes, fatty alcohol-based polyoxyalkylenes, fatty alcohol polyoxyalkylene alkyl ethers, and mixtures thereof. Surfactants, if used, will be used in an effective amount, and a typical effective amount is an amount up to 5% by weight (excluding any filler content).

Wetting agents also may be included in the curable composition. Wetting agent selection will depend on the application requirements and the resin chemistry utilized. Wetting agents, if used, will be used in an effective amount and a typical effective amount is up to 5% by weight (excluding any filler content). Examples of suitable wetting agents include Fluorad FC-4430 Fluorosurfactant available from 3M, Clariant Fluowet OTN, BYK W-990, Surfynol 104 Surfactant, Crompton Silwet L-7280, Triton X100 available from Rhom and Haas, Propylene glycol with a preferable Mw greater than 240, Gama-Butyrolactone, castor oil, glycerin or other fatty acids, and silanes.

A flow control agent also may be included in the curable composition. Flow control agent selection will depend on the application requirements and resin chemistry employed. Flow control agents, if used, will be present in an effective amount: an effective amount is an amount up to 5% by weight (excluding any filler content). Examples of suitable flow control agents include Cab-O-Sil TS720 available from Cabot, Aerosil 8202 or R972 available from Degussa, fumed silicas, fumed aluminas, or fumed metal oxides.

Some curable compositions may include an adhesion promoter, and selection of an appropriate adhesion promoter will depend on the application requirements and resin chemistry employed. Adhesion promoters, if used, will be used in an effective amount and an effective amount is an amount up to 5% by weight (excluding any filler content). Examples of suitable adhesion promoters include: silane coupling agents such as Z6040 epoxy silane or Z6020 amine silane available from Dow Corning; A186 Silane, A187 Silane, A174 Silane, or A1289 available from OSI Silquest; Organosilane SI264 available from Degussa; Johoku Chemical CBT-1 Carbobenzotriazole available from Johoku Chemical; functional benzotriazoles; thiazoles; titanates; and zirconates.

An air release agent (defoamer) is another optional component to the curable composition. Air release agent selection will depend on the application requirements and resin chemistry employed. Air release agents, if used, will be used in an effective amount and an effective amount will be an amount up to 5% by weight (excluding any filler content). Examples of suitable air release agents include Antifoam 1400 available from Dow Corning, DuPont Modoflow, and BYK A-510.

In some embodiments these curable compositions are formulated with tackifying resins in order to improve adhesion and introduce tack; examples of tackifying resins include naturally-occurring resins and modified naturally-occurring resins; polyterpene resins; phenolic modified terpene resins; coumarons-indene resins; aliphatic and aromatic petroleum hydrocarbon resins; phthalate esters; hydrogenated hydrocarbons, hydrogenated rosins and hydrogenated rosin esters.

In some embodiments other components may be included in the curable composition, for example, diluents such as liquid polybutene or polypropylene; petroleum waxes such as paraffin and microcrystalline waxes, polyethylene greases, hydrogenated animal, fish and vegetable fats, mineral oil and synthetic waxes, naphthenic or paraffinic mineral oils.

In other embodiments, monofunctional reactive diluents can be included in the curable composition to incrementally delay an increase in viscosity without adversely affecting the physical properties of the cured coating. Suitable diluents include p-tert-butyl-phenyl glycidyl ether, allyl glycidyl ether, glycerol diblycidyl ether, glycidyl ether of alkyl phenol (commercially available from Cardolite Corporation as Cardolite NC513), and Butanediodiglycidylether (commercially available as BDGE from Aldrich).

Other additives, such as stabilizers, antioxidants, impact modifiers, and colorants, in types and amounts known in the art, may also be added to the curable composition.

Common solvents that readily dissolve the resins, and with a proper boiling point ranging from 25° C. to 200° C. can be used in the curable composition. Examples of solvents that may be utilized include ketones, esters, alcohols, ethers, and other common solvents that are stable. Suitable solvents include γ-butyrolactone, propylene glycol methyl ethyl acetate (PGMEA), and 4-methyl-2-pentanone.

Curing can take place by thermal exposure, ultraviolet (UV) or microwave irradiation, or a combination of these. Curing conditions will be tailored to the specific formulation and can be readily determined by the practitioner. Furthermore, the curable composition may be B-stageable or not, depending on the application requirements.

The compound and/or curable composition of this invention may be used as an adhesive, coating, or encapsulant. They are especially useful for electronic device construction within the semiconductor packaging industry because of their inherent temperature resistance as well as the ability to include functionalities to tailor such properties as adhesion, toughness, conductivity, melting point, high temperature modulus, and solubility. In one embodiment the curable composition is used to affix a semiconductor device onto a substrate or to encapsulate or coat parts, or all, of the device. The inventive compound and curable composition have particular utility for the bonding of integrated circuit chips (semiconductor dies) to substrates including glass, ceramic, organic, and metal leadframes. The inventive compound and curable composition are also useful for the bonding of circuit packages or assemblies to printed wire boards, the encapsulation of solder balls used as electrical connections, and the coating of via holes.

The compound of this invention is particularly useful in curable compositions for capillary underfill of flip-chip devices. In flip-chip technology, the active side of the semiconductor die is bumped with metallic solder balls and flipped so that the solder balls can be aligned and placed in contact with corresponding electrical terminals on the substrate. Electrical connection is realized when the solder is reflowed to form metallurgical joints with the substrates. The coefficients of thermal expansion (CTE) of the semiconductor die, solder, and substrate are dissimilar and this mismatch stresses the solder joints, which ultimately can lead to failure of the semiconductor package. Organic materials, often filled with organic or inorganic fillers or spacers, are used to underfill the gap between the die and the substrate to offset the CTE mismatch and to provide enforcement to the solder joints. Such underfill materials can be applied through a capillary effect, by dispensing the material along the edges of the die-substrate assembly after solder reflow and letting the material flow into the gap between the die and substrate. The underfill is then cured, typically by the application of heat.

One problem that is known in prior art capillary underfill formulations is that of “shrinkage voids”. This phenomenon, which is often seen in acrylate-based underfill formulations, is caused by the shrinkage of the underfill during cure and results in a void, or gap, between the underfill and either the solder balls, substrate, semiconductor die, or a combination of these. Such voids are undesirable, as they can ultimately lead to device failure. The compound of this invention may be designed to be an oligomer that has a high molecular weight and fewer reaction points compared to a monomer. Such a compound will not shrink as much during cure and will give superior shrinkage void performance compared to, for instance, acrylate monomers. The use of a maleimide functionality that has a high glass transition temperature (Tg) will help retain a high Tg in the overall underfill formulation. High Tg is desirable for underfill formulations, as it allows the underfill to remain below the glass transition temperature during the thermal cycling of the device, and this, in turn, reduces stress in the assembly.

The compound of this invention may also be used in wafer backside coating applications. Typically, wafer backside coating is a printable, B-stage-able adhesive formulation that is coated on the backside of a semiconductor wafer by screen or stencil printing. After printing, the coated wafer is heated to evaporate solvent and/or partially advance the resin, so that the coating is hardened to a non-tacky state. The wafer is then laminated onto dicing tape, diced, and singulated into individual dies with an adhesive layer on the die backside. The die can then be attached to a substrate using heat and pressure. After die attach, the adhesive is typically cured in either a snap or oven cure process. Coatings comprising the compound of this invention can give higher thermal conductivity for these types of applications as compared to prior art formulations. This attribute can be an advantage in many die attach applications.

### EXAMPLES

In the structures for these examples, m and n are integers that will vary with the proportion of starting materials, and typically will each be, independently, any integer within the range of 1 to 100, provided that n is greater than m. It will be understood by those skilled in the art that the level of substitution of the dependent hydrocarbon chains containing the ester and maleimide groups and groups other than maleimide on the DPO backbone can be calculated, although the exact location on the DPO backbone cannot be precisely determined.

### Example 1

### Preparation of Compound from Diphenyl Oxide (DPO), Paraformaldehyde, and Malemido Caproic Acid (MCA)

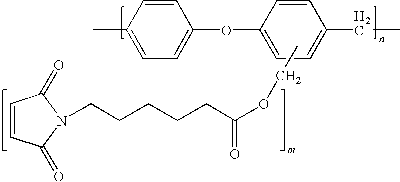

DPO (40.0 grams, 0.2350 mol), paraformaldehyde (22.4 grams), p-toluene sulfonic acid monohydrate (4.48 grams, 0.0118 mol), and maleimidocaproic acid (MCA) (109.0 grams, 0.2585 mol) were charged to a 250 mL 4-neck round-bottom reaction flask equipped with a condenser, thermometer and mechanical mixer. The flask was placed in an oil bath preheated to 130° C. and the contents heated with mixing at 300 rpm. The reaction temperature of 105° C. was achieved within 15 minutes, during which time, the reaction contents changed from an opaque gold mixture to a cloudy gold solution and a white sublimate formed on the flask sides. The reaction was kept within the temperature range of 105°-110° C. for one hour. At this point, the reaction turned cloudy orange-gold. The reaction was stopped and left to cool over night, which resulted in a mixture of crystalline solids and clear gold syrup. Next, the mixture was poured into 700 mL of swirling methanol, mixed well and left to sit over night.

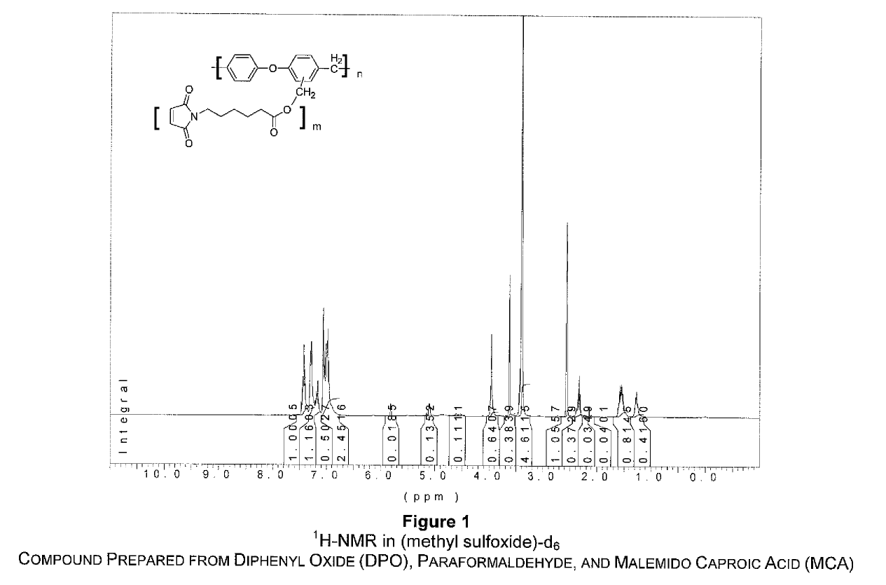

A light gold paste formed in a hazy-gold liquor. The paste was rinsed several times with methanol and then dissolved in 400 mL of dichloromethane. Insoluble flocculent was filtered out and the remaining clear yellow solution washed three times with 300 mL of distilled water. Emulsions were formed by the washes, which were broken with the addition of saturated sodium chloride solution. Following the washes, the bottom organic phase was collected as a hazy yellow solution and dried over 20 grams of magnesium sulfate. Filtration resulted in a clear yellow reaction solution that was charged to a 1 L 4-neck round-bottom reaction flask equipped with a mechanical mixer. An exchange resin (30 grams) was added, the mixture stirred vigorously for one hour and filtered. The filtration was very slow. Solvent was stripped from the solution on a roto-evaporator at 40° C. leaving a light gold paste. The oligomeric compound was characterized by ¹H-NMR, FIG. 1.

### Example 2

### Preparation of Compound from Diphenyl Oxide (DPO), Paraformaldehyde, MCA and Glacial Acetic Acid

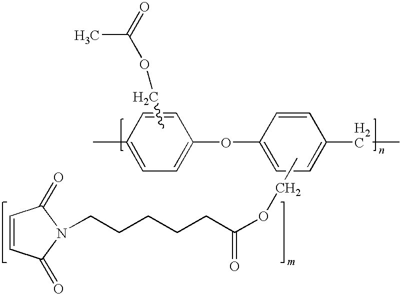

DPO (40.0 grams, 0.2350 mol), paraformaldehyde (22.4 grams), p-toluene sulfonic acid monohydrate (4.48 grams, 0.0118 mol), glacial acetic acid (15.5 grams, 0.2585 mol) and maleimidocaproic acid (MCA) (54.5 grams, 0.2585 mol) were charged to a 250 mL 4-neck round-bottom reaction flask equipped with a condenser, thermometer, and mechanical mixer. The flask was placed in an oil bath preheated to 130° C. and the contents heated with mixing at 300 rpm. The reaction temperature of 105° C. was achieved within 15 minutes, during which time, the reaction changed from an opaque gold mixture to a clear gold solution. The reaction was kept at 105-110° C. for a total of one hour. At this point, the reaction was cloudy orange-gold, and no sublimate was formed. The reaction was stopped and left to cool over night, which resulted in a mixture of crystalline solids and clear gold syrup. The mixture was poured into 700 mL of swirling methanol, mixed well and left to sit over night.

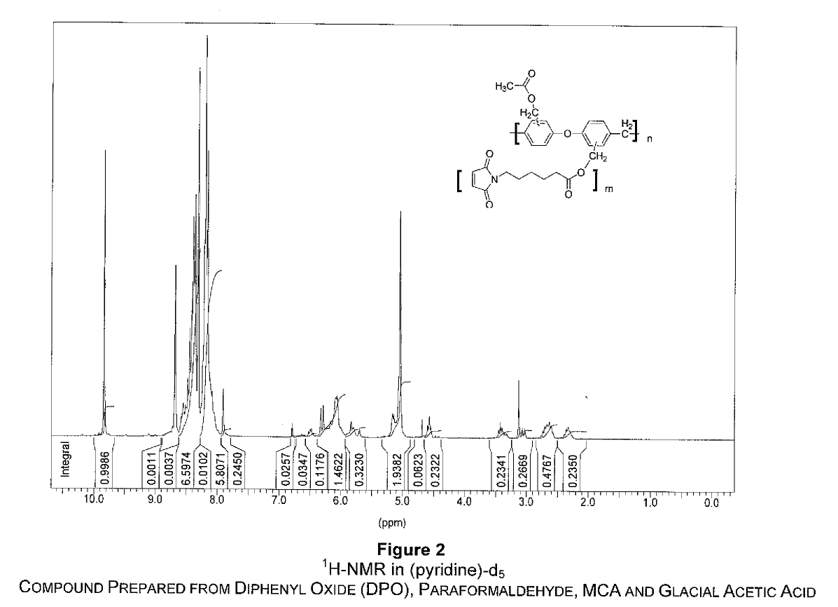

A light-colored coagulated mass formed in a hazy-gold liquor. The mass was rinsed several times with methanol and dissolved in 400 mL of dichloromethane. The clear yellow dichloromethane product solution was washed three times with 300 mL of distilled water. Emulsions formed by the washes were broken with the addition of saturated sodium chloride solution. Following the washes, the bottom organic phase was collected as a hazy yellow solution and dried over 20 grams of magnesium sulfate. Filtration resulted in a clear yellow reaction solution. This was charged to a 1 L 4-neck round-bottom reaction flask equipped with a mechanical mixer. An exchange resin (30 grams) was added, the mixture was stirred vigorously for one hour and filtered. Filtration was slow. Solvent was stripped from the solution on a roto-evaporator at 40° C. and a Kugfelrohr at 60° C. leaving 24 grams of a light gold paste. The oligomeric compound was characterized by ¹H-NMR, FIG. 2.

### Example 3

### Preparation of Compound from Diphenyl Oxide (DPO), Paraformaldehyde, MCA and 2-Ethyl Butyric Acid

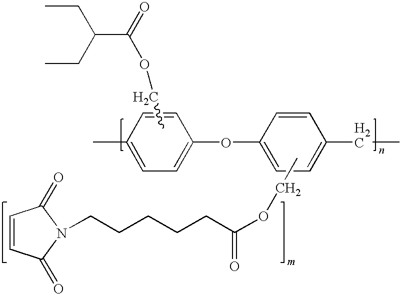

DPO (120 grams, 0.7050 mol), paraformaldehyde (67.2 grams), p-toluene sulfonic acid monohydrate (13.41 grams, 0.0705 mol), 2-ethyl butyric acid (90.08 grams, 0.7755 mol) and maleimidocaproic acid (MCA) (163.6 grams, 0.7755 mol) were charged to a 1 L 4-neck round-bottom reaction flask equipped with a condenser, thermometer and mechanical mixer. The flask was placed in an oil bath preheated to 130° C. and the contents heated with mixing at 300 rpm. The reaction temperature of 105° C. was achieved within 15 minutes. During this time, the reaction changed from a gold-colored to an orange-colored mixture. Some sublimate formed and was knocked back down into the reaction flask. The reaction was kept at 105°-112° C. for a total of one hour. At this point, the reaction was a clear gold solution with some white sublimate in the condenser. The reaction was stopped and left to cool.

The reaction mixture then was poured into 2100 mL of swirling methanol and mixed well for 10 minutes. A light ivory suspension formed. After the solids settled out, the methanol was observed to be a deep gold color and was decanted off. The solids were washed with methanol and decanted three more times. Residual methanol was removed via vacuum filtration leaving a tacky ivory cake with gold residue (37 grams moist). The cake was dissolved in dichloromethane resulting in 100 mL of a slightly hazy gold solution. The solution was added to 900 mL of methanol and mixed mechanically for 15 minutes. A white suspension formed and was allowed to settle for one hour. The methanol solution was decanted off the white solids and residual solvent was removed via vacuum filtration.

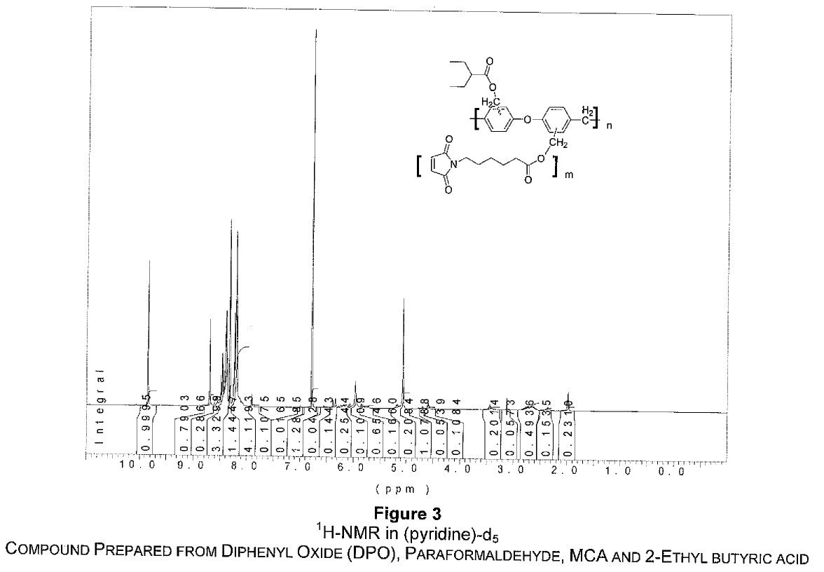

The solids were dissolved to 500 mL in dichloromethane in preparation for a water wash. The hazy yellow solution was then washed vigorously in a separatory funnel with 750 mL of a brine solution. The resulting emulsion was allowed to separate over night. The following morning, a clear yellow organic solution was collected from the funnel. The white aqueous layer was discarded. The organic solution was dried over magnesium sulfate, filtered and the volume was increased from 475 mL to 700 mL by the addition of dichloromethane. An exchange resin (90 grams) was added and the reaction mixed for one hour. The mixture was filtered to remove the resin, the resin was rinsed with dichloromethane and the dichloromethane added to the reaction mix. Filtration was slow. The solvent was stripped from the solution on a roto-evaporator at 40° C. leaving 16 grams of a gold waxy material, which was confirmed as the product using ¹H-NMR, FIG. 3.

### Example 4

### Preparation of Compound from Diphenyl Oxide (DPO), Paraformaldehyde, MCA and Phenylacetic Acid

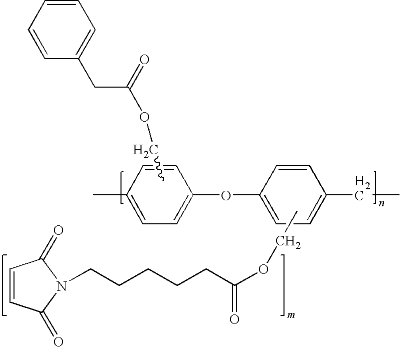

DPO (108.0 grams, 0.6344 mol), paratormaldehyde (60.2 grams), p-toluene sulfonic acid monohydrate (12.06 grams, 0.0634 mol), phenylacetic acid (95.0 grams, 0.6978 mol) and maleimidocaproic acid (MCA) (147.2 grams, 0.6978 mol) were charged to a 1 L 4-neck round-bottom reaction flask equipped with a condenser, thermometer and mechanical mixer. The flask was placed in an oil bath preheated to 130° C. and the contents heated with mixing at 300 rpm. Reaction temperature (105° C.) was achieved within 15 minutes. During this time, the reaction changed to a homogeneous orange mixture with a mild white sublimate. The reaction was kept at 105°-110° C. for a total of one hour. As the sublimate formed, it was knocked back down into the reaction. By the end of the heating time, the reaction was a clear orange solution. The reaction was stopped and left to cool. The reaction mixture was poured over five minutes into 1500 mL of swirling methanol and mixed well for ten minutes.

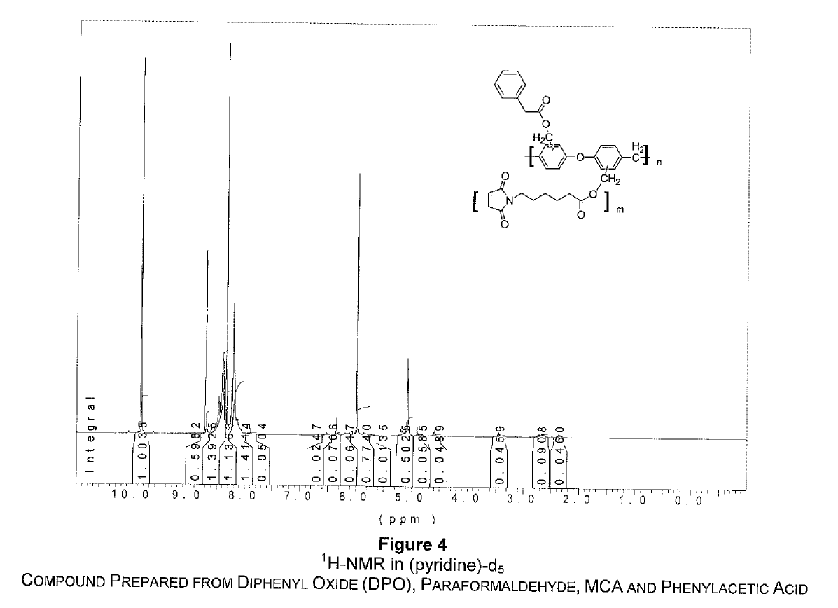

A light ivory suspension was formed. After the lumpy ivory solids settled out, the solvent, which had become a cloudy light gold, was decanted off. The solids were further washed with methanol and decanted three more times. The solids were dissolved in 250 mL of dichloromethane and this solution was added to 1500 mL of swirling methanol. Ivory-colored solids precipitated out and were allowed to settle for one hour. The methanol solution was decanted off the white solids. The solids were then dissolved in 500 mL in dichloromethane and filtered to remove light insolubles. The product solution was then washed vigorously in a separatory funnel with 1000 mL of a brine solution. The organic solution was collected from the funnel and the white aqueous layer was discarded. Next, the solution was dried over 40 grams of magnesium sulfate and filtered. The volume was increased to 700 mL with the addition of dichloromethane. An exchange resin (90 grams) was added and mixed for one hour. The mixture was filtered and the exchange resin rinsed with dichloromethane. Solvent was stripped from the hazy gold solution on a roto-evaporator at 40° C. leaving 26 grams of a white powder. The identity of the product was confirmed using ¹H-NMR, FIG. 4.

### Example 5

### Preparation of Adduct 6 from Diphenyl Oxide (DPO), Paraformaldehyde, MCA and Valeric Acid

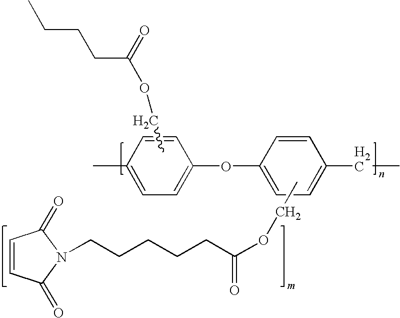

DPO (136.4 grams, 0.8011 mol), paraformaldehyde (76.1 grams), p-toluene sulfonic acid monohydrate (15.24 grams, 0.0801 mol), valeric acid (90.0 grams, 0.8812 mol) and maleimidocaproic acid (MCA) (185.9 grams, 0.8812 mol) were charged to a 1 L 4-neck round-bottom reaction flask equipped with a condenser, thermometer and mechanical mixer. The flask was placed in an oil bath preheated to 130° C. and the contents heated with mixing at 300 rpm. Reaction temperature (105° C.) was achieved within 15 minutes. During this time, the reaction changed to an opaque gold mixture and a mild white sublimate was noted. As the sublimate formed, it was knocked back down into the reaction. The reaction was kept at 105°-110° C. for a total of one hour resulting in a clear orange solution. The reaction was stopped and left to cool. Next, the reaction was poured over five minutes into 1800 mL of swirling methanol and mixed well for 10 minutes.

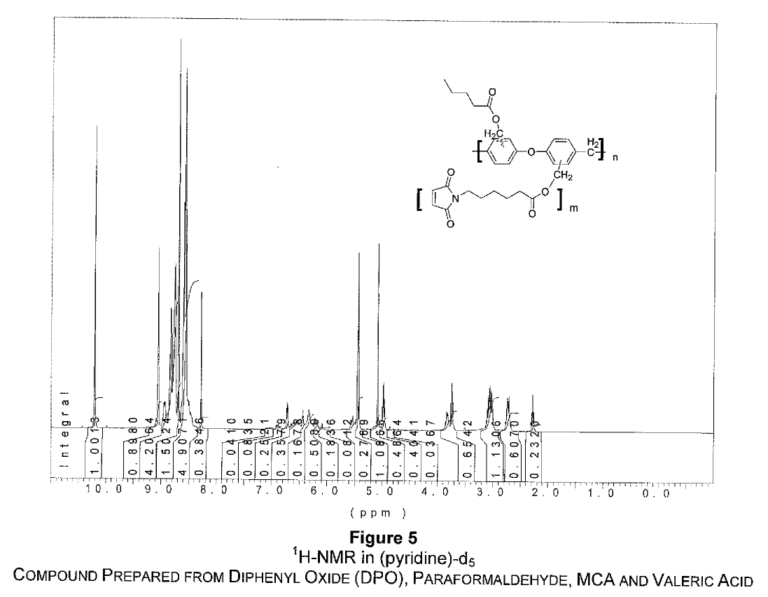

A light ivory suspension was formed. After the solids settled out, the solvent was decanted off. The solids were then washed with methanol and decanted three more times. Residual methanol was removed via vacuum filtration leaving a sticky light gold cake (75 grams moist). The cake was dissolved in 700 mL of dichloromethane. The product solution was washed vigorously in a separatory funnel with 1300 mL of a brine solution. The resulting emulsion was allowed to separate over 2.5 hours. The organic solution was collected from the funnel and the white aqueous layer was discarded. The solution was then dried over 40 grams of magnesium sulfate and filtered. The volume was increased to 700 mL with the addition of dichloromethane. An exchange resin (90 grams) was added and mixed for one hour. The mixture was filtered and the exchange resin rinsed with dichloromethane. Solvent was stripped from the hazy gold solution on a roto-evaporator at 40° C. leaving 40 grams of a light gold waxy material. The identity of the product was confirmed using ¹H-NMR, FIG. 5.

### Example 6

### Preparation of Compound from Diphenyl Oxide (DPO), Paraformaldehyde, MCA and Cyclohexylacetic Acid

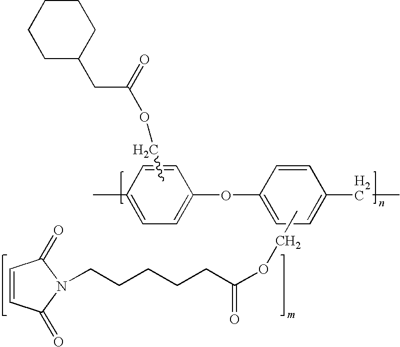

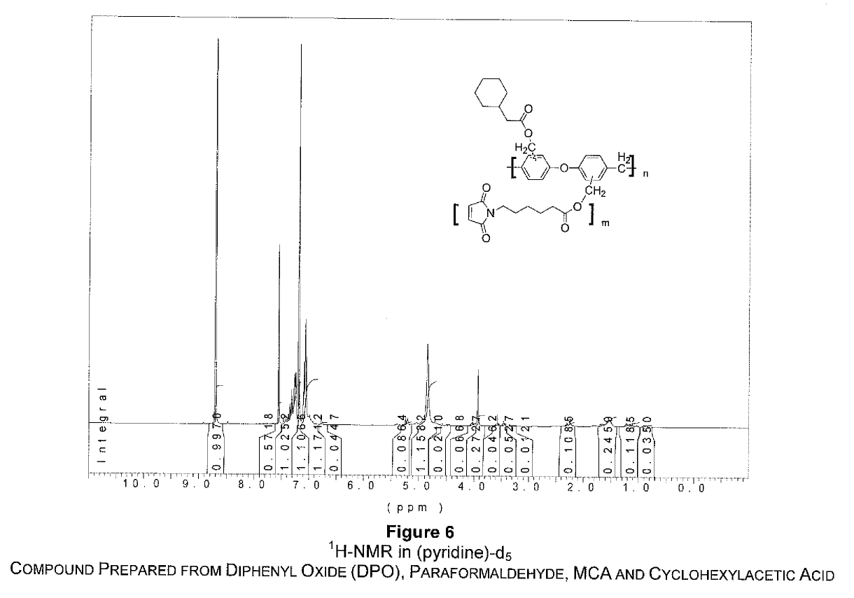

DPO (89.1 grams, 0.5237 mol), paraformaldehyde (49.7 grams), p-toluene sulfonic acid monohydrate (9.97 grams, 0.0524 mol), cyclohexylacetic acid (81.9 grams, 0.5761 mol) and maleimidocaproic acid (MCA) (121.6 grams, 0.5761 mol) were charged to a 1 L 4-neck round-bottom reaction flask equipped with a condenser, thermometer and mechanical mixer. The flask was placed in an oil bath preheated to 130° C. and the contents heated with mixing at 300 rpm. Reflux was achieved within 15 minutes at 105° C. During this time, the reaction changed from a gold mixture to an opaque orange dispersion. Sublimate formed and was knocked down back into the reaction. The reaction was kept at 105°-110° C. for a total of one hour. At this point, the reaction was cloudy orange-gold. The reaction was stopped and left to cool. Next, the mixture was poured into 1600 mL of swirling methanol and mixed well for 10 minutes. A light ivory suspension was formed. After the solids settled out, the methanol was decanted off. The solids were then washed with 100 mL of methanol and decanted three more times. The oligomeric material was dried and then characterized by ¹H-NMR, FIG. 6.

What is claimed is:

1. A compound having a diphenyl oxide backbone, and pendant from the backbone at least one hydrocarbon chain, the hydrocarbon chain containing an ester functionality and being terminated with a maleimide functional group.

2. The compound of claim 1 having pendant from the backbone at least one additional hydrocarbon chain, the additional hydrocarbon chain containing an ester functionality and being terminated with a group other than a maleimide.

3. The compound of claim 2 wherein the additional functional group is selected from the group consisting of acrylate, methacrylate, maleate, fumarate, styrenic, cinnamyl, or a mixture of these.

4. A compound selected from the group consisting of: 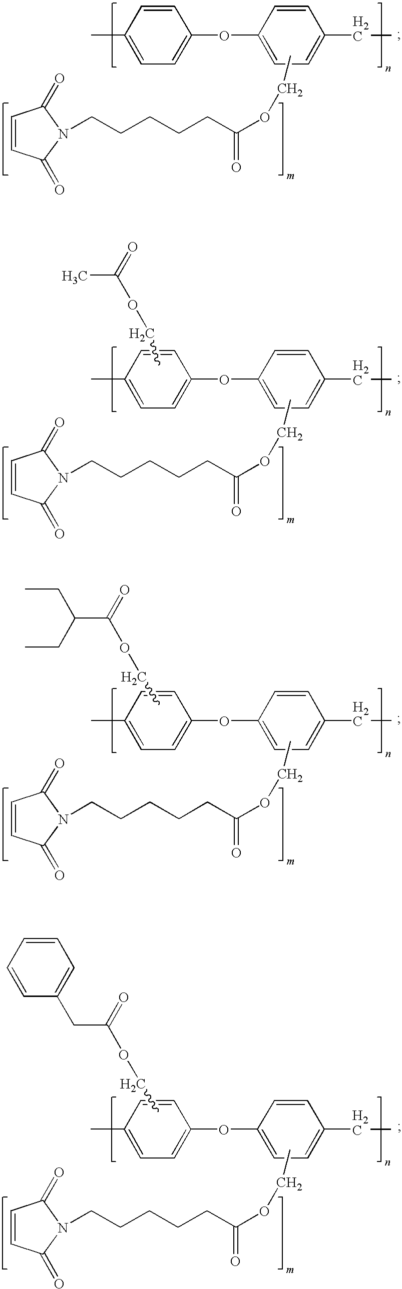 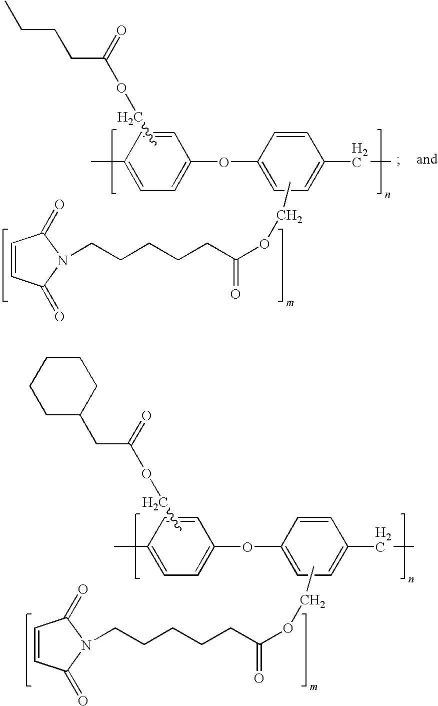
in which m and n are independently an integer from 1 to 100, provided that n is greater than m.

5. A process for making a semiconductor device comprising the steps of:
(i) providing a semiconductor wafer having a front side that is active and, opposed to the front side, a back side that is inactive,
(ii) providing a curable composition comprising a compound having a diphenyl oxide backbone, and pendant from the backbone at least one hydrocarbon chain, the hydrocarbon chain containing an ester functionality and being terminated with a maleimide functional group,
(iii) applying the curable composition to the back side of the semiconductor wafer to form an adhesive layer having an exposed side opposite the front side of the semiconductor wafer,
(iv) B-staging the adhesive layer to form a B-staged wafer,
(v) dicing the B-staged wafer into at least one semiconductor die having an adhesive layer on the back side of the semiconductor die,
(vi) contacting the semiconductor die to a substrate such that the adhesive layer is disposed between the semiconductor die and the substrate, and
(vii) curing the adhesive for a time and temperature to adhere the semiconductor die to the substrate to form a semiconductor device.

6. The process according to claim 5 in which the compound of the curable composition of (ii) further has pendant from the backbone at least one additional hydrocarbon chain, the additional hydrocarbon chain containing an ester functionality and being terminated with a group other than a maleimide.

7. A process for underfilling a flip chip semiconductor device comprising the steps of:
(i) providing a flip chip die that has been attached to a substrate such that there is a gap between the flip chip die and the substrate,
(ii) dispensing a curable composition comprising a compound having a diphenyl oxide backbone, and pendant from the backbone at least one hydrocarbon chain, the hydrocarbon chain containing an ester functionality and being terminated with a maleimide functional group onto the substrate along at least one side of the flip chip die,
(iii) allowing the curable composition to flow into the gap between the flip chip die and the substrate, and
(iv) curing the curable composition.

8. The process according to claim 7 in which the compound of the curable composition of (ii) further has pendant from the backbone at least one additional hydrocarbon chain, the additional hydrocarbon chain containing an ester functionality and being terminated with a group other than a maleimide.

9. A process for attaching a semiconductor die to a substrate comprising the steps of:
(i) providing a semiconductor die, a substrate, and a curable composition comprising a compound having a diphenyl oxide backbone, and pendant from the backbone at least one hydrocarbon chain, the hydrocarbon chain containing an ester functionality and being terminated with a maleimide functional group,
(ii) applying the curable composition to the substrate, the die, or both,
(iii) joining the semiconductor die to the substrate with the curable composition disposed between them, and
(iv) curing the curable composition.

10. The process according to claim 9 in which the compound of the curable composition of (i) further has pendant from the backbone at least one additional hydrocarbon chain, the additional hydrocarbon chain containing an ester functionality and being terminated with a group other than a maleimide.
